SEQUENCE LISTING (73 sequences)

SEQ ID NO: 1 - RNA, 23 nt, Homo sapiens
  source 1..23: mol_type = genomic RNA
tgaggggcagagagcgagacttt

SEQ ID NO: 2 - RNA, 22 nt, Homo sapiens
  source 1..22: mol_type = genomic RNA
taggtagtttcatgttgttggg

SEQ ID NO: 3 - RNA, 22 nt, Homo sapiens
  source 1..22: mol_type = genomic RNA
atgcacctgggcaaggattctg

SEQ ID NO: 4 - RNA, 22 nt, Homo sapiens
  source 1..22: mol_type = genomic RNA
cagcagcaattcatgttttgaa

SEQ ID NO: 5 - RNA, 17 nt, Homo sapiens
  source 1..17: mol_type = genomic RNA
gcggggctgggcgcgcg

SEQ ID NO: 6 - RNA, 22 nt, Homo sapiens
  source 1..22: mol_type = genomic RNA
tttccggctcgcgtgggtgtgt

SEQ ID NO: 7 - RNA, 22 nt, Homo sapiens
  source 1..22: mol_type = genomic RNA
tattgcactcgtcccggcctcc

SEQ ID NO: 8 - RNA, 23 nt, Homo sapiens
  source 1..23: mol_type = genomic RNA
aacattcaacgctgtcggtgagt

SEQ ID NO: 9 - RNA, 22 nt, Homo sapiens
  source 1..22: mol_type = genomic RNA
ccaggaggcggaggaggtggag

SEQ ID NO: 10 - RNA, 23 nt, Homo sapiens
  source 1..23: mol_type = genomic RNA
cccagtgttcagactacctgttc

SEQ ID NO: 11 - RNA, 22 nt, Homo sapiens
  source 1..22: mol_type = genomic RNA
tcaggccaggcacagtggctca

SEQ ID NO: 12 - RNA, 19 nt, Homo sapiens
  source 1..19: mol_type = genomic RNA
ttgatctcggaagctaagc

SEQ ID NO: 13 - RNA, 22 nt, Homo sapiens
  source 1..22: mol_type = genomic RNA
gcgggggtggcggcggcatccc

SEQ ID NO: 14 - RNA, 23 nt, Homo sapiens
  source 1..23: mol_type = genomic RNA
gaagtgcttcgattttggggtgt

SEQ ID NO: 15 - RNA, 18 nt, Homo sapiens
  source 1..18: mol_type = genomic RNA
cgggcgtggtggtggggg

SEQ ID NO: 16 - RNA, 22 nt, Homo sapiens
  source 1..22: mol_type = genomic RNA
gtgaggaggggctggcagggac

SEQ ID NO: 17 - RNA, 25 nt, Homo sapiens
  source 1..25: mol_type = genomic RNA
gaacccatgaggttgaggctgcagt

SEQ ID NO: 18 - RNA, 22 nt, Homo sapiens
  source 1..22: mol_type = genomic RNA
tctgggcaacaaagtgagacct

SEQ ID NO: 19 - RNA, 17 nt, Homo sapiens
  source 1..17: mol_type = genomic RNA
ctccgggacggctgggc

SEQ ID NO: 20 - RNA, 18 nt, Homo sapiens
  source 1..18: mol_type = genomic RNA
gtgaaggcccggcggaga

SEQ ID NO: 21 - RNA, 23 nt, Homo sapiens
  source 1..23: mol_type = genomic RNA
tcaagagcaataacgaaaaatgt

SEQ ID NO: 22 - RNA, 22 nt, Homo sapiens
  source 1..22: mol_type = genomic RNA
aggcggggcgccgcgggaccgc

SEQ ID NO: 23 - RNA, 18 nt, Homo sapiens
  source 1..18: mol_type = genomic RNA
gggtgcgggccggcgggg

SEQ ID NO: 24 - RNA, 22 nt, Homo sapiens
  source 1..22: mol_type = genomic RNA
ctgggagaaggctgtttactct

SEQ ID NO: 25 - RNA, 22 nt, Homo sapiens
  source 1..22: mol_type = genomic RNA
aatgcacctgggcaaggattca

SEQ ID NO: 26 - RNA, 23 nt, Homo sapiens
  source 1..23: mol_type = genomic RNA
taatccttgctacctgggtgaga

SEQ ID NO: 27 - RNA, 22 nt, Homo sapiens
  source 1..22: mol_type = genomic RNA
atagtccgagtaacgtcggggc

SEQ ID NO: 28 - RNA, 22 nt, Homo sapiens
  source 1..22: mol_type = genomic RNA
ctggtacaggcctgggggacag

SEQ ID NO: 29 - RNA, 21 nt, Homo sapiens
  source 1..21: mol_type = genomic RNA
accactgcactccagcctgag

SEQ ID NO: 30 - RNA, 22 nt, Homo sapiens
  source 1..22: mol_type = genomic RNA
ttctcaagagggaggcaatcat

SEQ ID NO: 31 - RNA, 22 nt, Homo sapiens
  source 1..22: mol_type = genomic RNA
ttctggaattctgtgtgaggga

SEQ ID NO: 32 - RNA, 22 nt, Homo sapiens
  source 1..22: mol_type = genomic RNA
aacacacctattcaaggattca

SEQ ID NO: 33 - RNA, 21 nt, Homo sapiens
  source 1..21: mol_type = genomic RNA
gatctcactttgttgcccagg

SEQ ID NO: 34 - RNA, 23 nt, Homo sapiens
  source 1..23: mol_type = genomic RNA
agcagcattgtacagggctatca

SEQ ID NO: 35 - RNA, 23 nt, Homo sapiens
  source 1..23: mol_type = genomic RNA
tggggggacagatggagaggaca

SEQ ID NO: 36 - RNA, 22 nt, Homo sapiens
  source 1..22: mol_type = genomic RNA
taggtagtttcctgttgttggg

SEQ ID NO: 37 - RNA, 22 nt, Homo sapiens
  source 1..22: mol_type = genomic RNA
aaaccgttaccattactgagtt

SEQ ID NO: 38 - RNA, 22 nt, Homo sapiens
  source 1..22: mol_type = genomic RNA
taacagtctacagccatggtcg

SEQ ID NO: 39 - RNA, 19 nt, Homo sapiens
  source 1..19: mol_type = genomic RNA
tggatttttggatcaggga

SEQ ID NO: 40 - RNA, 22 nt, Homo sapiens
  source 1..22: mol_type = genomic RNA
ttgtacatggtaggctttcatt

SEQ ID NO: 41 - RNA, 21 nt, Homo sapiens
  source 1..21: mol_type = genomic RNA
tacagtactgtgataactgaa

SEQ ID NO: 42 - RNA, 22 nt, Homo sapiens
  source 1..22: mol_type = genomic RNA
tggcagtgtcttagctggttgt

SEQ ID NO: 43 - RNA, 21 nt, Homo sapiens
  source 1..21: mol_type = genomic RNA
cagcagcacactgtggtttgt

SEQ ID NO: 44 - RNA, 21 nt, Homo sapiens
  source 1..21: mol_type = genomic RNA
ctatatatcaaacatattcct

SEQ ID NO: 45 - RNA, 23 nt, Homo sapiens
  source 1..23: mol_type = genomic RNA
tagtgcaatattgcttatagggt

SEQ ID NO: 46 - RNA, 21 nt, Homo sapiens
  source 1..21: mol_type = genomic RNA
gtgcattgtagttgcattgca

SEQ ID NO: 47 - RNA, 22 nt, Homo sapiens
  source 1..22: mol_type = genomic RNA
ttcactggagtttgtttcaata

SEQ ID NO: 48 - RNA, 22 nt, Homo sapiens
  source 1..22: mol_type = genomic RNA
tgagacctctgggttctgagct

SEQ ID NO: 49 - RNA, 22 nt, Homo sapiens
  source 1..22: mol_type = genomic RNA
cctcccacacccaaggcttgca

SEQ ID NO: 50 - RNA, 22 nt, Homo sapiens
  source 1..22: mol_type = genomic RNA
tgatatgtttgatatattaggt

SEQ ID NO: 51 - RNA, 21 nt, Homo sapiens
  source 1..21: mol_type = genomic RNA
taattttatgtataagctagt

SEQ ID NO: 52 - RNA, 22 nt, Homo sapiens
  source 1..22: mol_type = genomic RNA
aacccgtagatccgatcttgtg

SEQ ID NO: 53 - RNA, 23 nt, Homo sapiens
  source 1..23: mol_type = genomic RNA
tgagtgtgtgtgtgtgagtgtgt

SEQ ID NO: 54 - RNA, 24 nt, Homo sapiens
  source 1..24: mol_type = genomic RNA
tgaggggcctcagaccgagctttt

SEQ ID NO: 55 - RNA, 22 nt, Homo sapiens
  source 1..22: mol_type = genomic RNA
actggacttggagtcagaaggc

SEQ ID NO: 56 - RNA, 23 nt, Homo sapiens
  source 1..23: mol_type = genomic RNA
agctcggtctgaggcccctcagt

SEQ ID NO: 57 - RNA, 23 nt, Homo sapiens
  source 1..23: mol_type = genomic RNA
aggaagccctggaggggctggag

SEQ ID NO: 58 - RNA, 20 nt, Homo sapiens
  source 1..20: mol_type = genomic RNA
ggctccttggtctaggggta

SEQ ID NO: 59 - RNA, 18 nt, Homo sapiens
  source 1..18: mol_type = genomic RNA
cggtgagcgctcgctggc

SEQ ID NO: 60 - RNA, 22 nt, Homo sapiens
  source 1..22: mol_type = genomic RNA
actcggcgtggcgtcggtcgtg

SEQ ID NO: 61 - RNA, 22 nt, Homo sapiens
  source 1..22: mol_type = genomic RNA
cgcatcccctagggcattggtg

SEQ ID NO: 62 - RNA, 22 nt, Homo sapiens
  source 1..22: mol_type = genomic RNA
tttgttcgttcggctcgcgtga

SEQ ID NO: 63 - RNA, 17 nt, Homo sapiens
  source 1..17: mol_type = genomic RNA
gggagaagggtcggggc

SEQ ID NO: 64 - RNA, 17 nt, Homo sapiens
  source 1..17: mol_type = genomic RNA
gtgggggagaggctgtc

SEQ ID NO: 65 - RNA, 22 nt, Homo sapiens
  source 1..22: mol_type = genomic RNA
gtgaacgggcgccatcccgagg

SEQ ID NO: 66 - RNA, 19 nt, Homo sapiens
  source 1..19: mol_type = genomic RNA
aatggatttttggagcagg

SEQ ID NO: 67 - RNA, 22 nt, Homo sapiens
  source 1..22: mol_type = genomic RNA
tgcggggctagggctaacagca

SEQ ID NO: 68 - RNA, 25 nt, Homo sapiens
  source 1..25: mol_type = genomic RNA
actggacttggagtcagaagagtgg

SEQ ID NO: 69 - RNA, 23 nt, Homo sapiens
  source 1..23: mol_type = genomic RNA
cccactgccccaggtgctgctgg

SEQ ID NO: 70 - RNA, 23 nt, Homo sapiens
  source 1..23: mol_type = genomic RNA
agctggtgttgtgaatcaggccg

SEQ ID NO: 71 - RNA, 22 nt, Homo sapiens
  source 1..22: mol_type = genomic RNA
aactgtttgcagaggaaactga

SEQ ID NO: 72 - RNA, 21 nt, Homo sapiens
  source 1..21: mol_type = genomic RNA
ctagactgaagctccttgagg

SEQ ID NO: 73 - RNA, 17 nt, Homo sapiens
  source 1..17: mol_type = genomic RNA
ggcgggtgcgggggtgg

(19) United States
(12) Patent Application Publication
(10) Pub. No.: US 2025/0034657 A1
(43) Pub. Date: Jan. 30, 2025

(54) METHOD FOR SCREENING TUBERCULOSIS-INFECTED SAMPLE AND PROBE SET TO BE USED THEREFOR

(71) Applicant: KANSAI MEDICAL UNIVERSITY EDUCATIONAL CORPORATION, Osaka (JP)

(72) Inventors: Seiji KANDA, Osaka (JP); Toshimasa NISHIYAMA, Osaka (JP); Takaki SHIMONO, Osaka (JP)

(73) Assignee: KANSAI MEDICAL UNIVERSITY EDUCATIONAL CORPORATION, Osaka (JP)

(21) Appl. No.: 18/716,263

(22) PCT Filed: Dec. 20, 2022
(86) PCT No.: PCT/JP2022/046903

§ 371 Date: Jun. 4, 2024

(30) Foreign Application Priority Data
Dec. 21, 2021 (JP) 2021-207106

(51) Int. Cl. C12Q 1/689

(52) U.S. Cl. CPC C12Q 1/689; C12Q 2600/158; C12Q 2600/178

(57) ABSTRACT

A method of screening for the presence or absence of tuberculosis infection using a urine specimen and a probe set for the screening are provided. The method uses a ratio [B/S] of content B of biomarker miRNA (sequence ID2 to ID33 and/or ID: 34 to ID: 73) to content S of hsa-miR-423-5p present in a urine-derived sample as an index. Comparing with the cut-off value based on the corresponding ratios N (B/S) for healthy population and P [B/S] for tuberculosis infected population, and the urine-derived sample is classified into a tuberculosis infection-positive or negative based on the comparison result.

### TECHNICAL FIELD

The present invention relates to a screening method for classifying a large number of specimen samples into possibly positive for tuberculosis infection and possibly negative for tuberculosis infection, and more particularly, relates to a screening method which can easily classify urine specimen samples at a reduced cost per specimen, and a probe set used for the screening.

### BACKGROUND

One quarter of the total world population is said to be infected with tuberculosis, and the infection is spreading particularly in developing countries. For this reason, it is desired to develop a method capable of quickly and simply determining the presence or absence of tuberculosis infection, which can be carried out even in developing countries.

As a method for diagnosing tuberculosis infection, QuantiFERON (registered trademark) TB and T-Spot (registered trademark) TB) for measuring interferon γ (INF-γ) produced from lymphocytes by antigen stimulation with ESAT-6, CFP-10 or the like are generally used in our country. Such methods are assessment in which blood is collected from a subject, lymphocytes are separated, a certain amount thereof is dispensed into a culture plate coated with an anti-human interferon γ antibody, then Mycobacterium tuberculosis specific antigens ESAT-6 and CFP-10 are added and cultured for about 20 hours, and then the number of INF-γ-producing cells bound to the anti-human INF-γ antibody is measured. It is not affected by BCG inoculation or non-tuberculous mycobacteria, and thus has a characteristic of high specificity as compared with the tuberculin reaction. However, these methods take about 2 days for determination due to the culture. In addition, these methods require a device for cell separation and cell culture, and thus require a high cost per specimen. For these reasons, these methods are not commonly employed in developing countries.

In addition, assessment based on the measurement of INF-γ has a problem concerning a patient infected with not only Mycobacterium tuberculosis but also HIV (human immunodeficiency virus), called as “co-infected patients with HIV and tuberculosis”. As the number of INF-γ-producing cells in co-infected patients with HIV and tuberculosis is too low for the detection, in the case of immunodeficiency, resulting in a false negative determination for tuberculosis infection.

On the other hand, an MPB64 antibody is known as an antibody specific to a patient with active tuberculosis, and there is also known a method for determining whether or not a patient is in a state of active tuberculosis by detecting an amount of the MPB64 antibody in plasma using the antibody as a marker, for example, by dot blotting. It has been reported that the MPB64 antibody is also present in urine and correlates with results of using serum as a measurement sample (Non-Patent Document 1: Yasuko Tamada et. al., Microbiol immunol 2012; 56:740-747). In an assessment using the MPB64 antibody as a marker, urine can be used as a measurement sample which is easily collected. As the method can show the determination of the presence or absence of infection quickly, the method is useful in a developing country where tuberculosis infection is likely to spread. However, the presence of an antibody showing a cross-reaction is also known, and a BCG vaccinated person may be classified into false positive in the assessment.

Assessment of sputum culture from a subject to determine the presence or absence of Mycobacterium tuberculosis is reliable. However, since it requires a specialized equipment and a culture period of 1 to 4 weeks, it is not suitable for a rapid screening/diagnosis.

Under such circumstances, as an assessment for the presence or absence of tuberculosis infection in developing countries, a desired screening/diagnosis method should rapidly provide results without the need for cell culture. Additionally, it should minimize errors such as false positives or false negatives, even when utilizing less invasive specimens.

In recent years, it has been proposed to use a miRNA as a biomarker other than antibodies, cells, cytokines, and the like. Abnormal expression of miRNAs has been discovered in the development and malignant alteration of many diseases, and miRNAs can also be detected from body fluids such as blood, and therefore. These facts suggest that the use of miRNAs in liquid biopsies is suitable.

The use of miRNAs as diagnostic markers has been variously reported in recent years, mainly for cancer (U.S. Pat. No. 5,156,829 (various cancers: Patent Document 1), U.S. Pat. No. 6,489,583 (pancreatic cancer: Patent Document 2), WO2019/244575 (gastric cancer: Patent Document 3)).

In Patent Document 2, two types of miRNAs are specified as biomarkers, and the abundance of the biomarker miRNAs present in a blood sample is compared with the abundance of the respective miRNAs in a healthy/normal subject to determine the possibility of pancreatic cancer onset. The two types of miRNAs are a combination of a miRNA whose abundance increases and a miRNA whose abundance decreases when the possibility of pancreatic cancer onset is high as compared with those in healthy/normal subjects, and the use of such a combination enhances the determination accuracy of the possibility of pancreatic cancer onset. For comparison of the abundances of the miRNAs with those in healthy/normal subjects, a ΔCt value (obtained by subtracting a Ct value of cel-miR39 contained in a measurement sample from a Ct value of the sample) after correction using cel-miR39 as an external control is used. The differentiation between positive and negative is performed by comparison with a cut-off value calculated based on the ΔCt value for the biomarker miRNAs.

Patent Document 3 proposes a method for determining morbidity of gastric cancer using an expression level of a miRNA identified as a gastric cancer biomarker serving an index. For the determination of the expression level, a specific miRNA (hsa-miR3610 or hsa-miR4669) is used as a normalization factor, and a value (ΔCt value) obtained by subtracting a Ct value of the normalization factor from a Ct value of the biomarker is used.

In addition, JP 2021-90451 A (Patent Document 4) proposes that a less invasive sample (serum) is used to identify a specific piRNA (a non-coding RNA molecule longer than a miRNA (26 to 31 nucleotides)) as a biomarker. The piRNA is used for a screening method for classifying samples into positive and negative for colorectal cancer. In this proposed method, an expression level of the marker RNA is compared with a reference expression level.

The expression level used for the comparison is an expression level normalized with miR93-5p that is a endogenous control, and the cut-off value for differentiation and determination can be set by statistical analysis of the normalized expression levels in a healthy/normal population and a intestine patients population.

Also, in patients with tuberculosis, some unique miRNAs have been reported (Non-Patent Document 2: Naveed Sabir et. al., Frontiers in Microbiology vol. 9, March 2018), and Non-Patent Document 2 introduces, in Table 1, miRNAs as candidates for biomarkers when human peripheral blood, whole blood, serum, or a macrophage is used as a specimen. However, the miRNAs shown in Table 1 are diverse depending on the reference documents in the field that human peripheral blood or serum is used as a specimen. It is unclear which miRNAs have what degrees of specificity or how to determine their potential as diagnostic markers.

In addition, blood is an excellent liquid biopsy, however, drawing blood for collecting a specimen sample is necessary. The assessment using a blood-derived sample is conducted only for patients who are strongly suspected of having tuberculosis infection in developing countries. This is a limitation in application as an assessment for widely screening patients infected with tuberculosis.

### PRIOR ART

### Patent Document

[Patent Document 1] JP 5156829 B
[Patent Document 2] JP 6489583 B
[Patent Document 3] WO2019/244575
[Patent Document 4] JP 2021-90451 A

### Non-Patent Document

[Non-Patent Document 1] Yasuko Tamada et. al., “Diagnosis of active tuberculosis using MBP64, a specific antigen of Mycobacterium bovis”, Microbiol immunol 2012; 56:740-747
[Non-Patent Document 2] Naveed Sabir et. al., “miRNAs in Tuberculosis: New Avenues for Diagnosis and Host-Directed Therapy”, Frontiers in Microbiology vol. 9, March 2018

### SUMMARY OF THE INVENTION

### Technical Problem to be Solved by the Invention

An object of the present invention is to provide a screening method that can obtain useful information on the presence or absence of tuberculosis infection in a short time and at low cost, by detecting a biomarker from a less invasive liquid biopsy, particularly urine which is an easily collected specimen sample. The object of the invention is also to provide a probe set for the screening.

### Means for Solving the Problem

The present inventors have comprehensively detected and analyzed miRNAs contained in urine collected from patients with active tuberculosis and from healthy/normal subjects, identified miRNAs that are specifically enhanced/reduced in expression in patients infected with tuberculosis as compared with healthy/normal subjects, and specified as candidates of biomarker miRNAs that can be used as an index for determination of tuberculosis infection, and filed a patent application (PCT/JP2021/023027).

The amount of a miRNA (miRNA concentration) present in urine specimen varies among individuals. Therefore, determination based on a result of the comparison in expression level (content) of the biomarker miRNA contained in a given urine specimen to the same miRNA in a urine specimen from a healthy/normal subject is unreliable.

With respect to an expression frequency of the miRNA, comparison of the biomarker miRNA present in the given sample with an average expression frequency of healthy/normal subjects to determine whether the expression of the biomarker miRNA is enhanced/reduced can avoid the problem raised from a concentration difference between individuals. However, in order to evaluate the magnitude in the expression frequency of a specific miRNA, accurate amounts of miRNAs must be obtained for a population parameter. In other words, identifying and quantifying a large number of miRNAs present in each individual was required for obtaining the total amount of present miRNAs. Such measurement can be done with an expensive device such as a next generation sequencer which can simultaneously identify and quantify several hundred or more kinds of miRNAs, and requires a longer time for analysis per individual sample. Therefore, the method is not utilized for a large-scale screening method like a health examination in which a sample having a risk of tuberculosis infection is picked from a large number of samples.

In general, a miRNA used as a biomarker does not have so high expression frequency (about 0 to 2.5%). For using a particular miRNA as a biomarker whose expression frequency becomes lower than that of healthy/normal subjects due to tuberculosis infection, the total amount as the population parameter needs to be determined with accuracy. From the viewpoint of improving the accuracy concerning the total amount, many kinds of miRNAs should be detected. However, in order to meet the requirements, an RNA array including various types of primer probes, an expensive PCR apparatus, and a lot of measurement technicians and analysts are required.

The present inventors have further studied a method for determining whether the expression of the miRNA used as the biomarker is enhanced or reduced as compared with that in a healthy/normal subject without obtaining the total amount (total number of reads) of a large number of miRNAs expressed in each individual. Then, various studies were conducted on a miRNA serving as criteria (internal control as a standard) for identifying miRNAs serving as biomarkers for tuberculosis infection is increasing or decreasing in each individual.

A miRNA serving an internal control standard must satisfy the following criteria:
a) miRNA is constantly expressed across individuals, and has a highly detectable level of expression in each individual,
b) miRNA display small variation in expression frequency among individuals, and
c) miRNA is minimally affected by tuberculosis infection.

The present inventors identified miRNA satisfying the above criteria for a standard miRNA as the internal control, and discovered that the presence or absence of tuberculosis infection can be determined by using as an index the relative content of the marker miRNA to the standard miRNA as this internal control, they have completed the present invention.

The method for screening a tuberculosis-infected sample of the present invention (Type I) is a method performed by detecting one or more biomarker miRNAs having a sequence selected from sequences of ID: 2 to ID: 33 and/or sequences of ID: 34 to ID: 73, contained in a given urine-derived sample from a subject.

The screening method type I comprises
obtaining a ratio [B/S] of content B of a biomarker miRNA to content S of hsa-miR-423-5p (sequence ID: 1) present in the given urine-derived sample;
comparing the obtained ratio [B/S] of the given urine-derived sample with a cut-off value, wherein the cut-off value is a value based on N (B/S) and P [B/S], wherein the N (B/S) represents a ratio of content B of the biomarker miRNA to content S of hsa-miR-423-5p in a urine-derived sample from a healthy/normal subject while P [B/S] represents the corresponding ratio in a urine-derived sample from a tuberculosis infected subject; and
classifying the given urine-derived sample into either a tuberculosis infection-positive or a tuberculosis-negative based on the comparison result.

The cut-off value is a ratio (B/S) that can statistically differentiate between a population of healthy/normal individuals and a population of individuals infected with tuberculosis. The cut-off value of B/S is set based on the ratio P (B/S) of the population of tuberculosis-infected individuals and the ratio of N (B/S) of an average of the population of healthy/normal individuals.

Where at least one member selected from the group consisting of hsa-miR-532-3p (sequence ID: 49), hsa-miR-423-3p (sequence ID: 56), and hsa-miR-451a (sequence ID: 37) is used as the biomarker, the given urine-derived sample with a [B/S] ratio lower than the cutoff value is classified as tuberculosis-infected sample during comparing process.

The classification involves two sorting steps, a first and a second sorting steps. In the first step, sorting is done using a first miRNA with a sequence chosen from ID: 2 to ID: 33 as the biomarker. In the second step, sorting is done using a second miRNA with a sequence chosen from ID: 34 to 73 as the biomarker. When a urine-derived sample in question is sorted into a tuberculosis infected sample in both the first and second sorting steps, the urine-derived sample can be determined as positive for tuberculosis.

According to another aspect of the invention, a method (Type II) for screening tuberculosis-infected samples comprises
measuring respective contents of hsa-miR-423-5p, and at least two biomarker miRNAs selected from the group consisting of hsa-miR-532-3p (sequence ID: 49), hsa-miR-423-3p (sequence ID: 56) and hsa-miR-451a (sequence ID: 37), in a given urine-derived sample from a subject;
obtaining a ratio [B/S] of each content B of the biomarker miRNAs to content S of the hsa-miR-423-5p; and
comparing the obtained ratios [B/S] of the given urine-derived sample, with a cut-off value for a respective ratio of the each biomarker wherein the cut-off value is a value obtained based on N (B/S) and P [B/S], wherein the N (B/S) represents a ratio of content B of the biomarker miRNA to content S of hsa-miR-423-5p in a urine-derived sample from a healthy/normal individual while P [B/S] represents the corresponding ratio in a urine-derived sample from a tuberculosis infected individual;
determining the given urine-derived sample as a tuberculosis infection-positive if at least one or all results of the comparison step has a [B/S] lower than the respective cut-off value.

The determination as the tuberculosis-infected sample in the type II screening method consists of only one sorting step corresponding to the second sorting step in the type I screening method. However, the determination may secure accuracy by conducting the comparison step for multiple biomarkers.

A probe set used for screening a tuberculosis-infected sample of the present invention comprises
a standard nucleotide probe capable of hybridizing to hsa-miR-423-5p under stringent conditions and labeled for detection; and
a probe selected at least one from the group consisting of nucleotide probe 1 and probe 2 below:
Probe 1: a nucleotide probe capable of hybridizing to one or more miRNAs represented by a sequence selected from ID2 to ID33 under stringent conditions and labeled for detection; and
Probe 2: a nucleotide probe capable of hybridizing to one or more miRNAs represented by a sequence selected from ID34 to ID73 under stringent conditions and labeled for detection.

For conducting the Type I screening for a tuberculosis infection sample, both of Probe 1 and Probe 2 are used.

For conducting the type II screening for tuberculosis-infected sample, a probe set containing a combination of said standard nucleotide probe and probe 3 described below is used.
Probe 3: a set of marker nucleotide probes, each of which can hybridize to a miRNA represented by a sequence ID: 49 or ID: 56 under stringent conditions and each labelled for detection.

The term “tuberculosis”, as used herein, refers to an infection with Mycobacterium tuberculosis. It refers to a state of active tuberculosis in which symptoms such as cough and weight loss have already appeared and Mycobacterium tuberculosis is detected from sputum. On the other hand, determination as to the presence or absence of latent tuberculosis, in which one is infected with Mycobacterium tuberculosis though having no abnormality in clinical findings such as symptoms, chest X-ray findings, and bacteriological findings, is not targeted.

The term “healthy subject”, as used herein, refers to a specimen derived from a person who is negative for tuberculosis infection in which Mycobacterium tuberculosis is not detected in sputum and can be recognized as AIDS-negative from the proportion of CD4-positive T cells.

As used herein, the “miRNA (microRNA)”, when specified by sequence ID (sequence No), refers to a miRNA specified by the sequence.

Unless otherwise specified, it is a concept including not only a mature miRNA which is a non-coding RNA of about 16 to 25 bases, but also a pri-miRNA which corresponds to an initial transcript obtained by transcription of an miRNA gene by RNA polymerase II and has a hairpin loop structure, and a pre-miRNA which corresponds to a precursor of the miRNA in a state in which the pri-mRNA is partially cleaved by RNaseIII-like Drosha.

As used herein, the “miRNA content” refers to the amount of a miRNA contained in a measurement sample, and can be usually quantified by real-time PCR. It includes both an absolute amount obtained by absolute quantification and a relative amount corresponding to an amount ratio [B/S] between a content B of a biomarker miRNA and a content S of a standard miRNA which is an endogenous control. The miRNA used as the endogenous control is present constitutively in patient as well as healthy subject, there is no significant difference in expression between the patient and healthy subject. Also, the biomarker miRNA as well as the standard miRNA are contained in the same measurement sample. Therefore, according to the invention, the relative amounts can be compared between samples, and the relative amount corresponding to the ratio [B/S] is used as a parameter served for a discrimination index between positive and negative for tuberculosis infection. Thus, the relative amount as well as the absolute amount may be used.

The quantification of the relative amount by real-time PCR may be performed using either a calibration curve or a ΔΔCt.

As for the content ratio [B/S] for the parameter used herein, the ratio [B/S] of a non-particular patient infected with tuberculosis is represented by P (B/S), and the ratio [B/S] of a non-particular healthy or normal person is represented by N (B/S). In addition, the content B of each biomarker in the sample is represented by “B (abbreviation of miRNA)”. For example, in the case of using hsa-miR-532-3p as the biomarker, the content of the biomarker is represented by B532-3p. Then, the content ratio of a biomarker to the standard internal miRNA as an endogenous control is abbreviated as follows: the content ratio of the biomarker, [B532-3p/S] in the case of using hsa-miR-532-3p as the biomarker, for a tuberculosis infected subject is represented by P (532-3p), and for a healthy/normal subject is represented by N (532-3p).

The term “miRNA expression frequency”, as used herein, refers to a proportion of the number of reads of a target miRNA (for example, biomarker) to a total number of reads in a case of being read by a next-generation sequencer. This corresponds to a proportion (%) of a content of the target miRNA to a total content of miRNAs. The total content is a sum of number of miRNAs (usually, 200 to 500 miRNAs) each having a detectable amount contained in the sample.

As used herein, the “number of cycles” means the number of repetitions of PCR performed until the target miRNA can be detected, and the larger the value of the number of cycles (represented by “Ct” in the present specification), the lower the content of the target miRNA.

The phrase “capable of hybridizing under stringent conditions”, as used herein, means that a nucleotide has a complementary sequence to each target miRNA, or a sequence which 85% or more, preferably 90% or more, and more preferably 95% or more match the complementary sequence.

### Effect of the Invention

In the screening method for a sample infected with tuberculosis of the present invention, a specific number of miRNAs are used as biomarkers, and the content ratio [B/S] of the biomarkers to the endogenous control is used as a determination index. Therefore, it is not necessary to obtain the total amount of miRNAs for calculating the expression frequency, and variation between individuals can be minimal.

Therefore, the screening method of the present invention makes it possible to classify a large number of samples into a possibly positive and a possibly negative in a short time without using an expensive equipment such as a next generation sequencer.

Furthermore, since a specimen sample for measurement is urine that does not require for collection by a specialist, a rapid screening for a possibly positive sample from a large amount of specimens may be done with a limited resource.

### BRIEF DESCRIPTION OF THE DRAWINGS

FIG. 1 is a scatter diagram showing the expression levels of hsa-miR23-5p in all subjects measured in Examples.

FIG. 2 is a scatter diagram showing the normalized content ratio [B/S] of hsa-miR532-3p.

FIG. 3 is a scatter diagram showing the normalized content ratio [B/S] of hsa-miR423-3p.

FIG. 4 is a scatter diagram showing the normalized content ratio [B/S] of hsa-miR451a.

FIG. 5 is a scatter diagram showing the normalized content ratio [B/S] of hsa-miR532-3p obtained in Verification 3.

FIG. 6 is a scatter diagram showing the normalized content ratio [B/S] of hsa-miR423-3p obtained in Verification 3.

### EMBODIMENT FOR CARRYING OUT THE INVENTION

<Standard Internal miRNA>

The standard miRNA used for the screening method for a sample infected with tuberculosis of the present disclosure is hsa-miR423-5p which is contained in each individual specimen, and has a sequence represented by “UGAGGGGCAGAGAGCGAGACUUU” (ID: 1).

hsa-miR423-5p has been reported to be constantly expressed in various normal or pathological tissues (Eliana Bignotti et. al., “Identification of stably expressed reference small non-coding RNAs for microRNA quantification in high-grade serous ovarian carcinoma tissues”, Journal of Cellular and Molecular Medicine 2016 vol. 20, No 12, pp. 2341-2348, Cosmo Bio: RNAi Handbook 2nd Edition, https://www.cosmobio.co.jp/upfiles/catalog/pdf/catalog_11711.pdf). The hsa-miR423-5p is stably and constantly expressed and displays small variation in expression in urine specimens of both healthy/normal subjects and tuberculosis-infected subjects. The inventors confirmed this through an analysis of miRNA present in urine specimens using a next-generation sequencer and quantification by real-time PCR (expression frequency 0.0086-0.29%). Here, “stably expressed” means that it is always present in urine and can be detected.

### <Biomarker for Determination of Tuberculosis Infection>

miRNAs increased/decreased in expression in a state of active tuberculosis, as compared with those in a healthy subject, can be used as a biomarker in the screening method for a sample from tuberculosis infected subject of the present disclosure.

Specifically, miRNAs used as the biomarker are: a miRNA belonging to a first group, which is overexpressed as compared with a healthy subject, and has a sequence chosen from sequences in Table 1 (ID: 2 to ID: 33); and a miRNA belonging to a second group, which is decreased in expression as compared with a healthy subject, and has a sequence chosen from sequences in Table 2 (ID: 34 to ID: 73).

TABLE 1

| sequence | | |
|---|---|---|
| No. | name | base sequence |
| 2 | hsa-miR-196a-5p | uagguaguuucauguuguuggg |
| 3 | hsa-miR-500a-3p | augcaccugggcaaggauucug |
| 4 | hsa-miR-424-5p | cagcagcaauucauguuuugaa |
| 5 | hsa-miR-4508 | gcggggcugggcgcgcg |
| 6 | hsa-miR-1180-3p | uuuccggcucgcgugggugugu |
| 7 | hsa-miR-92b-3p | uauugcacucgucccggccucc |
| 8 | hsa-miR-181a-5p | aacauucaacgcugucggugagu |
| 9 | hsa-miR-4459 | ccaggaggcggaggagguggag |
| 10 | hsa-miR-199a-5p | cccaguguucagacuaccuguuc |
| 11 | hsa-miR-1972 | ucaggccaggcacaguggcuca |
| 12 | hsa-miR-7641 | uugaucucggaagcuaagc |
| 13 | hsa-miR-4787-5p | gcggggguggcggcggcauccc |
| 14 | hsa-miR-373-3p | gaagugcuucgauuuuggggugu |
| 15 | hsa-miR-1268a | cgggcguggugguggggg |
| 16 | hsa-miR-6858-5p | gugaggaggggcuggcagggac |
| 17 | hsa-miR-1273d | gaacccaugagguugaggcugca |
| | | gu |
| 18 | hsa-miR-1285-3p | ucugggcaacaaagugagaccu |
| 19 | hsa-miR-4497 | cuccgggacggcugggc |
| 20 | hsa-miR-6126 | gugaaggcccggcggaga |
| 21 | hsa-miR-335-5p | ucaagagcaauaacgaaaaaugu |
| 22 | hsa-miR-663a | aggcggggcgccgcgggaccgc |
| 23 | hsa-miR-4466 | gggugcgggccggcgggg |
| 24 | hsa-miR-30c-2- | cugggagaaggcuguuuacucu |
| | 3p | |
| 25 | hsa-miR-502-3p | aaugcaccugggcaaggauuca |
| 26 | hsa-miR-500a-5p | uaauccuugcuaccugggugaga |
| 27 | hsa-miR-6723-5p | auaguccgaguaacgucggggc |
| 28 | hsa-miR-150-3p | ccgguacaggccugggggacag |
| 29 | hsa-miR-1273g- | accacugcacuccagccugag |
| | 3p | |
| 30 | hsa-miR-514b-5p | uucucaagagggaggcaaucau |
| 31 | hsa-miR-1299 | uucuggaauucugugugaggga |
| 32 | hsa-miR-362-3p | aacacaccuauucaaggauuca |
| 33 | hsa-miR-1285-5p | gaucucacuuuguugcccagg |

TABLE 2

| sequence | | |
|---|---|---|
| No. | name | base sequence |
| 34 | hsa-miR-107 | agcagcauuguacagggcuau |
| | | ca |
| 35 | hsa-miR-6887-5p | uggggggacagauggagagga |
| | | ca |
| 36 | hsa-miR-196b-5p | uagguaguuuccuguuguuggg |
| 37 | hsa-miR-451a | aaaccguuaccauuacugaguu |
| 38 | hsa-miR-132-3p | uaacagucuacagccauggucg |
| 39 | hsa-miR-1290 | uggauuuuuggaucaggga |
| 40 | hsa-miR-493-5p | uuguacaugguaggcuuucauu |
| 41 | hsa-miR-101-3p | uacaguacugugauaacugaa |
| 42 | hsa-miR-34a-5p | uggcagugucuuagcugguugu |
| 43 | hsa-miR-497-5p | cagcagcacacugugguuugu |
| 44 | hsa-miR-190a-5p | cuauauaucaaacauauuccu |
| 45 | hsa-miR-454-3p | uagugcaauauugcuuauaggg |
| | | u |
| 46 | hsa-miR-33a-5p | gugcauuguaguugcacugca |
| 47 | hsa-miR-653-3p | uucacuggaguuuguuucaaua |
| 48 | hsa-miR-769-5p | ugagaccucuggguucugagcu |
| 49 | hsa-miR-532-3p | ccucccacacccaaggcuugca |
| 50 | hsa-miR-190a-5p | ugacauguuugauauauuaggu |
| 51 | hsa-miR-590-3p | uaauuuuauguauaagcuagu |
| 52 | hsa-miR-99a-5p | aacccguagauccgaucuugug |
| 53 | hsa-miR-574-5p | ugagugugugugugugagugug |
| | | u |
| 54 | hsa-miR-3184-5p | ugaggggccucagaccgagcuu |
| | | uu |
| 55 | hsa-miR-378a-3p | acuggacuuggagucagaaggc |
| 56 | hsa-miR-423-3p | agcucggucugaggccccucag |
| | | u |
| 57 | hsa-miR-671-5p | aggaagcccuggaggggcugga |
| | | g |
| 58 | hsa-miR-4448 | ggcuccuuggucuaggggua |
| 59 | hsa-miR-4792 | cggugagcgcucgcuggc |
| 60 | hsa-miR-1307-3p | acucggcguggcgucggucgug |
| 61 | hsa-miR-324-5p | cgcauccccuagggcauuggug |
| 62 | hsa-miR-375-3p | uuuguucguucggcucgcguga |
| 63 | hsa-miR-4516 | gggagaagggucggggc |
| 64 | hsa-miR-1275 | gugggggagaggcuguc |
| 65 | hsa-miR-887-3p | gugaacgggcgccaucccgagg |
| 66 | hsa-miR- 1246 | aauggauuuuuggagcagg |
| 67 | hsa-miR-744-5p | ugcggggcuagggcuaacagca |
| 68 | hsa-miR-378c | acuggacuuggagucagaagag |
| | | ugg |
| 69 | hsa-miR-324-3p | cccacugccccaggugcugcug |
| | | g |
| 70 | hsa-miR-138-5p | agcugguguugugaaucaggcc |
| | | g |
| 71 | hsa-miR-452-5p | aacuguuugcagaggaaacuga |
| 72 | hsa-miR-1512-3p | cuagacugaagcuccuugagg |
| 73 | hsa-miR-3656 | ggcgggugcgggggugg |

These miRNAs are observed to have a significant difference in expression frequency as compared with an average profile of miRNAs contained in a urine sample from a healthy subject, through comprehensive detection of miRNAs contained in a urine sample from a tuberculosis infected subject. The miRNAs of the first group are observed to be significantly enhanced in expression as compared with the expression frequency in healthy subjects, and the miRNAs of the second group are observed to be significantly decreased in expression.

Here, the term “significant” in the level of significance indicates that the expression frequency is increased (miRNAs in Table 1) or decreased (miRNAs in Table 2) as compared with the expression frequency in a healthy subject at p<0.1 (preferably p<0.05) in Welch's t test or p<0.05 (preferably p<0.01) in the statistical processing by Fischer's exact establishment test regarding the analysis results of an RNA sequence using the next generation sequencer. Alternatively, two times or more (Table 1) or half or less (Table 2) expression frequency at the level of significance p<0.05 (preferably p<0.01) based on the statistical processing by the likelihood ratio test or the pseudo likelihood F test is also a significant case.

In addition, with respect to a content of the interest miRNA by a measurement with a real-time PCR, a case that the content is increased (Table 1) or decreased (Table 2) relative to an average content in a healthy subject at p<0.05 (preferably p<0.01) in the Mann Whitney U test is also a significance level one.

Among miRNAs included in the second group of the biomarker, hsa-miR-532-3p (sequence ID: 49), hsa-miR-423-3p (sequence ID: 56), and hsa-miR-451a (sequence ID: 37) are preferably used due to relatively high expression frequency. These miRNAs can be amplified for detection with low error rate even when using a urine specimen having a low miRNA concentration.

While only one member chosen from the above-mentioned miRNAs may be used alone as a biomarker, using a combination of two or more members is preferred. Utilizing multiple miRNAs can improve the determination accuracy based on the marker.

Using a combination of miRNAs from the first and second marker groups is particularly preferred. These groups demonstrate opposite expression due to tuberculosis infection: one group increases while the other decreases in expression frequency in tuberculosis infected subject. Therefore, employing a combination of biomarkers from the both groups can yield more reliable determination results (positive or negative for tuberculosis). In addition, even in the case of HIV infection, the risk of erroneous determination such as false positive or false negative can be lessened.

In addition, where a biomarker chosen from only either the first or second group for determination, the reliability of the determination results remains high if the determination based on all the comparison results consistent across multiple biomarkers used. For example, where the hsa-miR-532-3p (ID: 49) and hsa-miR-423-3p (ID: 56) in combination or a combination of three types miRNAs (i.e. hsa-miR-451a (ID: 37) in addition to the above-mentioned two miRNAs) is used as the biomarkers included in the second group, the number of specimen samples determined as false positive can be lessened.

### <Test Probe Set>

The test probe set of the present invention is a probe set for determining the possibility of positive for active tuberculosis for a urine-derived sample from a subject. The test prove comprises probes for the detection of the biomarker, specifically, comprises an internal standard probe for a standard miRNA (hsa-miR-423-5p) and a probe 1 for a biomarker selected from the first group and/or a probe 2 (preferably a probe 3) for a biomarker selected from the second group.

### (1) Internal Standard Probe

Probe capable of hybridizing to hsa-miR-423-5p under stringent conditions

An internal standard probe has a sequence complementary or 85% or more, preferably 90% or more, and more preferably 95% or more match to the base sequence (UGAGGGGCAGAGAGCGAGACUUU) of hsa-miR-423-5p represented by the sequence ID: 1, and is labeled for detection. If necessary, the sequence may be modified at 5′ end or 3′ end with a primer sequence, a linking nucleotide chain for immobilization to a carrier, an adaptor, and the like. In addition, a phosphate moiety and a sugar moiety of the nucleotide probe may be optionally modified.

### (2) Probe 1

Nucleotide probe or probe set capable of hybridizing under stringent conditions to one or more members from the first miRNA group (sequences ID: 2 to ID: 33) listed in Table 1 and labeled for detection

### (3) Probe 2

Nucleotide probe or probe set capable of hybridizing under stringent conditions to one or more members from the second miRNA group (sequences ID: 34 to ID: 73) listed in Table 2 and labeled for detection

A preferable probe 2 can hybridize under stringent conditions to at least one member selected from miRNAs consisting of the sequences ID: 37, ID: 49, and ID: 56, and labeled for detection.

### (4) Probe 3

The probe 3 is a preferable embodiment of the probe 2, and is a set of nucleotide probes for marker miRNAs ID: 49 and ID: 56. These probes can hybridize to miRNAs under stringent conditions and are labeled for detection.

The probe 3 may further contain a nucleotide probe which can hybridize to miRNA ID: 37 under stringent conditions and are labeled for detection.

The probe 1, the probe 2, and the probe 3 each has a sequence capable of hybridizing to a target miRNA under stringent conditions, and, may be modified with a primer sequence, a linking nucleotide chain for immobilization to a carrier, an adaptor, and the like according to needs, moreover, a phosphate moiety and a sugar moiety of the nucleotide probe may be optionally modified.

The nucleotide capable of hybridizing under stringent conditions refers to, herein, a nucleotide having a sequence complementary to each the target miRNA, or a nucleotide having a sequence 85% or more, preferably 90% or more, more preferably 95% or more, and particularly preferably 99% or more matching the complementary sequence.

The probe set of the present disclosure contains a combination of an internal standard probe and a biomarker probe. The combination of the probe set is shown below:
Internal standard probe and probe 1
Internal standard probe and probe 2
Internal standard probe, probe 1 and probe 2
Internal standard probe and probe 3
Internal standard probe, probe 1 and probe 3

The type of the probe for a biomarker is selected according to the type of screening method to be applied, that is, depending on differentiating process.

The probe set may be provided as a microarray, a probe panel, or the like.

The nucleotide probe hybridized to a target miRNA can be detected and quantified based on a label. The label may be either a direct or indirect label known to be used for a nucleotide probe. Examples of a direct label include isotopes such as ³²P. Examples of an indirect label include an antibody, an enzyme such as avidin-biotin, a chemiluminescent system such as luminescence/quenching, a fluorescent dye, and a gold colloid.

### <Preparation of Specimen and Measurement Sample>

The specimen to be tested (analyte) is urine collected from a subject. Unlike blood, urine among human body fluids does not require a specific technician to collect a specimen sample from subjects. Saliva as a specimen sample can be also collected without a specific technician, but saliva may not be easily collected from a dry mouse subject such as an elderly person. In this respect, urine is excellent as a specimen sample that can be easily collected from all subjects.

Therefore, the screening method of the present disclosure is suitable as a simple screening method for comprehensively evaluating a large number of samples.

Urine tends to contain less exosomes serving miRNAs than those contained in blood. However, urine can be collected in a large amount as compared with other body fluids, and, further, has no risk of infection with a blood-borne viral infection because of unnecessity for a blood sampling device such as a syringe, which is free from an accidental needle stick or the like. Therefore, urine as a specimen is useful to serve an analyte in a screening method performed in developing countries.

Like other body fluids such as blood, urine contains exosomes containing mature miRNAs used for biomarkers. The exosome is one of membrane vesicles secreted from cells, and generally has a diameter of 30 to 150 nm by electron microscope observation, and includes pri-miRNA, pre-miRNA, coding-mRNA, DNA, an enzyme, a cytoskeletal protein, a signal molecule, and the like in addition to the mature miRNA.

Urine collected from a subject may be utilized as it is, but a preferable sample for measurement is prepared from urine and contains nucleotides from the viewpoint of detecting the target miRNA by hybridization.

Examples of the sample derived from urine include a fraction in which exosomes are concentrated or roughly purified (hereinafter, referred to as “exosome rich fraction”), and RNA-containing samples extracted from the fraction. Among them, from the viewpoint of determination accuracy, a sample obtained by directly extracting RNAs is preferably used as the measurement sample.

RNA isolates as a measurement sample may be prepared from the urine by a conventionally known method.

For example, the exosome may be separated from a urine specimen by size exclusion chromatography, centrifugation, or the like, and an RNA may be extracted from the obtained exosome-rich fraction, or the exosome may be separated using a commercially available kit, or an RNA may be directly isolated.

As commercially available isolation kits, there are ExoQuick™ Exosome precipitation solution series (e.g., ExoQuick-TC, ExoQuick-CG, etc., both manufactured by System Biosciences) as an exosome isolation kit, a separation kit for obtaining nucleic acid samples extracted from urine which includes miRNeasy mini kit (manufactured by Qiagen), MagMax mirVana Total RNA kit (Thermo Fisher), Magtration (registered trademark) (Precision System Science) using magnetic particles.

Alternatively, used may be a method (nano-biodevice method) for collecing exosomes merely by delivering an exosome-containing sample (urine) to a nanowire structure.

A preferable sample contains all RNAs extracted from urine by, for example, conducting the operation with MagMax™ mirVana Total RNA-Seq kit (Thermo Fisher) in accordance with the protocol of its operation manual.

<Detection and Quantification of miRNA>

A method for detecting and quantifying target miRNAs (hsa-miR-423-5p and a biomarker miRNA) from a measurement sample (RNA-containing sample) prepared as described above, comprises performing hybridization with use of the probe set of the present disclosure, and measuring a label used in the probe, such as luminescence intensity, radioactivity, fluorescence intensity, and enzyme activity, based on the type of label.

Examples of the quantification of a miRNA include a real-time PCR, a microarray, Northem blotting, liquid phase nucleic acid hybridization, and a colorimetric method. Means for detecting a label through color development by a combination of a PCR and an immunochromatography may be used.

The means is appropriately employed depending on the type of label contained in the probe. For example, in the case of using a fluorescent label, detection can be performed by visually observing the presence or absence of fluorescence emitted by hybrid formation. In addition, the content and the content ratio can be calculated by scaling the intensity depending on the type of label, such as the fluorescence intensity.

Means for the quantification in the screening method of the present disclosure is means for quantification of the absolute amount as well as the relative amount of the target miRNAs contained in the measurement sample, without limitation.

As will be described later, the miRNA quantification is done for obtaining a ratio [B/S] of the content B of the biomarker miRNA to the content S of the hsa-miR-423-5p. Therefore, any techniques which can directly obtain the [B/S] without the calculation of the number of cycles, or without the contents of the hsa-miR-423-5p (endogenous control) and the target biomarker miRNA contained in the sample may be employed.

For example, when quantification is performed by the real-time PCR method, the [B/S] of the measurement sample can be obtained by either a calibration curve or the ΔΔCt.

### <Screening Method for Active Tuberculosis Samples>

The screening method of the present disclosure is a screening method for a possibly positive for active tuberculosis, and can be adopted as a screening method for classifying a large number of specimens into a positive sample and a negative sample as in a medical examination. The screening method of a simple assessment may also be used as an initial diagnosis on a patient suspected of having tuberculosis.

The screening method of the present disclosure is characterized by using a ratio [B/S] of the content B of the biomarker miRNA to the content S of the hsa-miR-423-5p contained in the specimen sample as an index for determining whether positive or negative.

That is, the screening method comprises:
detecting and quantifying hsa-miR-423-5p and a biomarker miRNA, which are present in a given sample prepared from a urine of a subject to be tested, to obtain a ratio [B/S] of content B of the biomarker miRNA to content S of the hsa-miR-423-5p; and
comparing the obtained [B/S] with a cut-off value for N (B/S) which is a corresponding ratio B/S of a sample prepared from urine from a healthy subject by a similar treatment method, and, based on the comparison result, classifying the given urine-derived sample into a tuberculosis infection-negative or positive depending on the group type of the biomarker (i.e. biomarker from first group or second group).

The [B/S] for the subject may be obtained as follows. The hsa-miR-423-5p (content S) and the selected biomarker miRNA (content B), which are present in the measurement sample, are detected and quantified using the above-mentioned detection/quantification method, and thereafter calculating the [B/S] for the biomarker of the measurement sample. Like a ΔΔCt method, the ratio [B/S] may be directly calculated based on the number of cycles (Ct) of the measurement value obtained by the real-time PCR.

In the case of using the probe set of the present disclosure, the [B/S] can be directly obtained, for example, by measuring specific intensities of the respective label.

The cutoff value is a threshold value for the [B/S] which can statistically distinguish between a population of healthy/normal subjects and a population of tuberculosis infected subjects, and may be appropriately set by ROC analysis or the like.

The cutoff value can be obtained by, for example, creating an ROC curve for a population data on the [B/S] for tuberculosis patients normalized by an average value of N (B/S). The average value is based on a population consisting of a large number of healthy/normal subjects. The threshold set as the cutoff value may be determined so that most of tuberculosis patients are included in the ROC curve concerning the population of the tuberculosis patients. In this case, the cut-off value corresponds to how many times higher the biomarker's expression frequency is in the patient compared to that in a healthy/normal subject.

The average value of the N (B/S) for normalization is obtained by detecting and quantifying the target marker miRNA for a population of healthy/normal subjects by the same one as that for the [B/S] of the measurement sample.

The cut-off value used for comparison is appropriately set depending on the biomarker miRNA, means for preparing a measurement sample, the type of label, means for quantification and so on.

The processes for obtaining and comparing the [B/S] for a measurement sample are performed according to each biomarker. Where two or more biomarkers are used, the [B/S] obtained for each biomarker is compared with the respective cut-off value, and, based on the rate to the cut-off value, the measurement sample is classified into positive or negative for tuberculosis infection.

In the case of selecting a miRNA listed in Table 1 (first miRNA group) as a first biomarker, if the obtained [B/S] is higher than the cut-off value for respective biomarker, the sample is classified into s sample infected with tuberculosis (first sorting).

In the case of selecting a miRNA listed in Table 2 (second miRNA group) as a second biomarker, if the obtained [B/S] is lower than the cut-off value, the sample is classified into a sample infected with tuberculosis (second sorting).

Where two or more biomarkers are used, each biomarker is compared with the respective cut-off value.

The screening method of the present disclosure may conduct either the first or second sorting step. However, for enhanced determination accuracy, conducting both the first and second sorting steps using biomarkers from their respective groups (type I screening method) is preferred.

When using a biomarker selected from either the first or second group, only one of the first or second sorting step is performed. In such case, using multiple biomarkers from either the first or second group ensures determination accuracy. A positive or negative determination is made only when all comparisons between the B/S value of each biomarker and the cut-off value match, thus ensuring determination accuracy.

In the case of performing both the first and second sorting steps, the order of these sorting steps is not particularly limited. The second sorting step may be performed after the first sorting step, or the first sorting step may be performed after the second sorting step. In addition, the first and second sorting steps may be performed simultaneously.

Multiple sorting may be performed simultaneously by using the probe set of the disclosure.

As a screening method using a biomarker selected from either the first or second group, there is a type II screening method in which a probe set comprising of an internal standard probe and a probe 3 in combination is used.

In the type II screening method, a sample is determined as positive for tuberculosis infection where the B/S values of all biomarkers used in probe 3 are lower than their respective cut-off values during the comparison process, thereby ensuring accurate determination.

However, if the B/S of any one of the biomarkers is below its cut-off value, categorizing the sample into positive for tuberculosis infection can reduce the number of false negatives.

The entire contents described in PCT/JP2021/023027 are incorporated herein by reference.

### EXAMPLES

Hereinafter, the present invention will be described more specifically with reference to Examples, but the present invention is not limited to the following Examples as long as it does not fall beyond the gist thereof.

### [Specimen]

Urine specimens from 19 Laotian healthy subjects, 19 Laotian patients infected with active tuberculosis (2 of them were co-infected with HIV), and 12 Japanese patients with severe COVID-19 (without tuberculosis infection) were used.

The urine specimens were collected and then cryopreserved. The urine specimens from the active tuberculosis patients were served to preparation of RNA-containing samples for measurement after a storage period of 1 year or more. The urine specimens from the healthy subjects and the serious COVID-19 patients were cryopreserved for a period within 60 days.

### [Preparation of RNA-Containing Sample for Measurement]

### (1) Preparation Method 1

A sample containing all RNAs extracted from 3 ml of a urine specimen in accordance with the protocol of an operation manual (urine sample) for MagMax (trademark) mirVana Total RNA-Seq kit (Thermo Fisher). Specific procedures are as follows.

To 3 ml of urine, 2.4 ml of Lysis Buffer was added, and the mixture was shaken for 7 minutes (500 rpm). To the resulting mixed liquid, 350 μl of Binding Beads Mix (mixed liquid of 234 μl of RNA Binding Beads and 116 μl of Lysis/Binding Enhancer) prepared separately was added, and the mixture was shaken for 5 minutes (500 rpm).

The resulting solution was added with 5.76 ml of isopropanol and pipetted, followed by incubation for 20 minutes (200 rpm). The solution was allowed to stand on a magnet stand to be separated, and then the supernatant liquid was discarded. RNAs were recombined on the RNA Binding Beads by adding a washing liquid, washing by shaking, and then adding a Turbo DNase™ solution, and the recombined RNAs were separated again by a magnet. After repeated separation and washing, an elution buffer was added to the dried RNA Binding Beads, followed by incubation at 65° C. for 5 minutes. The solution was allowed to stand on the magnet stand, and the resulting supernatant liquid was used as a measurement sample.

### (2) Preparation Method 2

An RNA extraction sample containing an miRNA was obtained from 15 ml of a urine specimen using Urine Conditioning Buffer (ZYMO RESEARCH), RNeasy Mini Kit and RNeasy MiniElute Cleanup Kit (Qiagen). Fifteen (15) ml of a urine specimen was pretreated (centrifuged (2000×g) at room temperature for 20 minutes). Then the supernatant was subjected to filtration with a PVDF membrane filter (MERCK) (Millex-GV Syringe Filter Unit, 0.22 μm) and concentration (by adding 1.05 μm of Urine Conditioning Buffer to the pretreated filtrate and centrifuging at room temperature for 20 minutes (3000×g)). Thus, a sample containing all extracted RNAs was obtained in accordance with the protocol for the RNeasy Mini Kit. Specific procedures are as follows.

Qiazol (2.25 ml) and isopropanol (450 μl) were added to and mixed with the precipitate obtained above, and the mixture was allowed to stand at room temperature for 2 minutes. Thereafter, the mixture was centrifuged at 4° C. for 30 minutes (maximum speed, 21100×g), the aqueous phase was added to the column attached to the RNeasy Mini Kit, and separation/washing by centrifugation was repeated (centrifugation at 8000×g or more at room temperature for 15 seconds→addition of flow through to RNeasy MiniElute Cleanup Kit→centrifugation at 8000×g or more at room temperature for 15 seconds (flow through is discarded)→addition of washing buffer attached to RNeasy MiniElute Cleanup Kit to column→centrifugation at 8000×g or more at room temperature for 15 seconds (flow through is discarded)→addition of 80% ethanol to column→centrifugation at 8000×g or more at room temperature for 2 minutes→centrifugation at room temperature at maximum speed of 21100×g for 5 minutes). Finally, 23 μl of sterilized water heated to 70° C. was added to the column, the mixture was allowed to stand at room temperature for 5 minutes, and then centrifuged (8000×g or more) at room temperature for 1 minute, and the obtained flow through liquid was used as a measurement sample.

### [Sequence Analysis Method]

To the RNA-containing sample obtained by Preparation method 1 or 2, an adaptor was added in accordance with the operation manual for Ion Total RNA-Seq kit v2 for small RNA Libraries (Thermo Fisher), and a reverse transcription reaction was performed to prepare a cDNA library for a next generation sequencer.

The prepared cDNA library was sequenced by the next generation sequencer Ion Torrent S5 (Thermo Fisher).

The obtained sequences were comprehensively collated using a known miRNA (miRNA registered in a data bank site: miRBase) as a reference sequence, the number of reads of each miRNA was calculated to quantify the miRNAs present in the sample, and the proportion of each miRNA to the total number of reads of the miRNAs was calculated.

In collation and identification with the reference sequence, when consecutive 10 bases or more matched, the miRNA was identified as the respective reference sequence.

Where the detected miRNAs were arranged in the order of expression frequency, it was possible to quantify 200 or more miRNAs from the prepared samples.

[Quantification of miRNA: Real-Time PCR]

For the measurement sample prepared from the urine specimen by Preparation method 2, the target miRNA content was quantified by qRT-real-time PCR. The quantitative process using the real-time PCR is as follows.

1. Reverse Transcription of miRNAs

Reverse transcription of the RNA-containing samples was performed using the miRCURY LNA miRNA PCR Starter Kit (Qiagen). For the reverse transcription reaction, a reaction solution (10 μl) having the following composition (volume μl per sample) was used in a 0.2 ml tube.

| | |
|---|---|
| 5 × miRCURY RT Reaction Buffer (buffer) | 2.0 μl |
| 10 × miRCURY RT Enzyme Mix (reverse transcriptase solution) | 1.0 μl |
| Template RNA | 6.5 μl |
| Nuclease-free water | 0.5 μl |

The reverse transcription reaction was performed under the following conditions using GeneAtlas G02 (ASTEC) which is a thermal cycler.
Step 1:42° C. for 60 min
Step 2:95° C. for 5 min
Step 3: stored at 4° C.

### 2. Real-Time PCR

### 2-1 Real-Time PCR Reaction (Using SYBR Green)

The cDNAs synthesized by the reverse transcription were quantified by the real-time PCR.

Real-time reactions were performed on 15-fold dilutions of synthesized CDNAs (dilution with nuclease-free water), 10 μl of the reaction solution (the following composition, the amount added is μl per sample) was prepared in a Strip Tube (0.1 ml) using the miRCURY LNA miRNA PCR Starter Kit (Qiagen), a real-time thermal cycler, Roter Gene Q (Qiagen) was used.

| | |
|---|---|
| 2 × miRCURY SYBR Green Master Mix (buffer) | 5.0 μl |
| PCR primer (miRCURY LNA miRNA PCR assay) | 1.0 μl |
| cDNA template (15-fold dilution) | 3.0 μl |
| Nuclease-free water | 1.0 μl |

In the PCR reaction, initial denaturation (95° C., 2 min) was performed, followed by 45 cycles of 95° C. for 10 seconds and 56° C. for 60 seconds. Melting curve analysis was performed at 1.0 sec/0.5° C. for the range of 60 to 95° C.

### 2-2 Calculation of Ct Value

For the Ct value, a threshold line serving as an auxiliary line was drawn in a region where an amplification curve of fluorescence was exponentially amplified, and an intersection point between the threshold line and the amplification curve was calculated as the Ct value.

2-3 Calculation of miRNA Content by Relative Quantification

A mixture obtained by mixing 1 μl of cDNA of each of the analyzed healthy subjects and patients was used as a reference sample.

Five types of standard samples obtained by serially diluting the reference sample 8 times, 32 times, 128 times, 512 times, and 2048 times were used to perform a real-time PCR reaction, thereby creating a calibration curve. The concentrations (relative values) of the standard samples are 256, 64, 16, 4, and 1, respectively.

### [Confirmation of Tuberculosis Infection and HIV Infection in Specimen]

The presence or absence of tuberculosis infection in the specimens subjected to the above sequence analysis was confirmed by culture inspection of Mycobacterium tuberculosis contained in the sputum of each subject and analysis by GeneXpert (registered trademark) system. The presence or absence of Mycobacterium tuberculosis by the GeneXpert (registered trademark) system was examined using a probe targeting a drug resistance region of the rpoB gene of Mycobacterium tuberculosis after amplification of RNA contained in the sputum by PCR.

With respect to the presence or absence of HIV infection, the presence or absence of HIV infection was examined by measuring the proportion of CD4 positive T cells using FACS Presto (registered trademark) from BD bioscience.

[Verification of Standard miRNA]

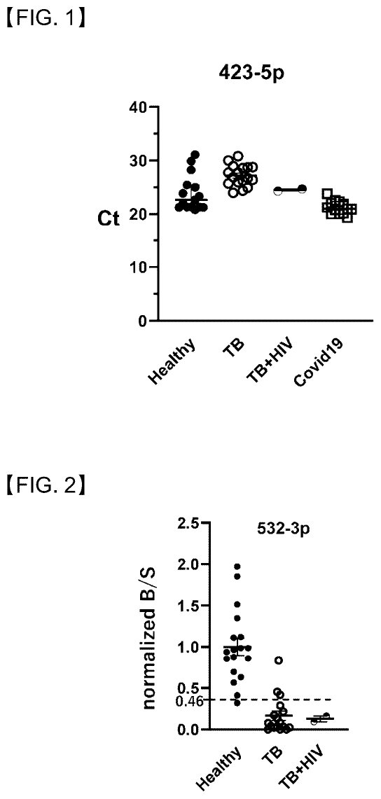

Measurement samples prepared by Preparation method 2 from urine specimens from a healthy subject, a patient infected with active tuberculosis, or a COVID-19 patient were identified and quantified by the real-time PCR method for hsa-miR-423-5p. The quantitation results are illustrated in FIG. 1.

In FIG. 1, the vertical axis represents Ct of hsa-miR-423-5p in each sample. Ct is the number of cycles when hsa-miR-423-5p was detected in each sample. A larger value of Ct indicates a lower content.

In FIG. 1, “Healthy” indicates a healthy subject, “TB” indicates a tuberculosis patient, “TB+HIV” indicates a co-infected subject with tuberculosis and HIV, and “COVID-19” indicates a Japanese serious COVID-19 patient.

It is understood that all the subjects in the tuberculosis infection-negative population (i.e. healthy subjects and COVID-19 patients) had a Ct value ranging from 20 to 24 with slight variation within the population. This suggests no significant difference in expression frequency of the hsa-miR-423-5p in urine among the races. In addition, it confirms that the individual difference within the population of tuberculosis negative subjects is minimal.

The Ct value for the TB population ranges from 25 to 30, and the variation within the population was small. Therefore, it is also understood that the hsa-miR-423-5p is a microRNA with a slight difference within the population of the tuberculosis positive individuals as well.

A difference in Ct value range was observed in the populations between the tuberculosis infection-negative subjects (Healthy and COVID-19) and the tuberculosis infection-positive subjects (TB and TB+HIV). This difference is considered due to a difference in cryopreservation period from collection of urine to the preparation of the measurement sample from the urine. The cryopreservation period was 60 days or less for the healthy subjects, while one year or more for the samples of the positive subject group depending on the type of sample. The target miRNAs present in the later sample may be more deteriorated due to a longer cryopreservation period.

From the above, it could be confirmed that the hsa-miR-423-5p is regularly present in a urine specimen with a slight difference among individuals and is not distinguishably affected in expression level due to HIV infection, tuberculosis infection or COVID-19 which is another respiratory disease. Therefore, the hsa-miR-423-5p is suitable as a standard miRNA for relative comparison.

### [Verification 1 of Biomarker by Screening Method of the Invention]

### (1) Setting of N (B/S)

For RNA-containing samples for measurement prepared from specimens of healthy subjects (Nos. 1 to 19) by Preparation Method 2, the contents of the standard internal microRNA (hsa-miR-423-5p), and hsa-miR-532-3p and hsa-miR-423-3p as the biomarkers selected from the second group were calculated based on the Ct value obtained from the calibration curve created using the real-time PCR. Next, the ratio [B/S] of the content (B) of each biomarker to the content(S) of the hsa-miR-423-5p was calculated. The measurement and calculation results for the healthy subjects are shown in Table 3.

TABLE 3

| healthy | | | | | | | |
|---|---|---|---|---|---|---|---|
| subject | content | | | B/S | | normalized B/S | |
| No. | 423-5p | 532-3p | 423-3p | 532-3p | 423-3p | 532-3p | 423-3p |
| 1 | 366.8 | 9627.5 | 301.5 | 26.24 | 0.82 | 0.94 | 1.00 |
| 2 | 366.3 | 15530.2 | 436.0 | 42.40 | 1.19 | 1.51 | 1.45 |
| 3 | 213.3 | 3775.9 | 114.1 | 17.70 | 0.54 | 0.63 | 0.65 |
| 4 | 347.5 | 10814.5 | 364.1 | 31.12 | 1.05 | 1.11 | 1.28 |
| 5 | 352.6 | 18314.3 | 283.9 | 51.94 | 0.80 | 1.85 | 0.98 |
| 6 | 31.9 | 768.5 | 26.2 | 24.07 | 0.82 | 0.86 | 1.00 |
| 7 | 60.4 | 1896.7 | 58.9 | 31.39 | 0.97 | 1.12 | 1.19 |
| 8 | 138.5 | 3319.2 | 31.6 | 23.97 | 0.23 | 0.86 | 0.28 |
| 9 | 341.9 | 9433.3 | 267.0 | 27.59 | 0.78 | 0.98 | 0.95 |
| 10 | 27.9 | 544.9 | 27.6 | 19.53 | 0.99 | 0.70 | 1.20 |
| 11 | 31 | 84.3 | 3.1 | 27.61 | 1.03 | 0.99 | 1.25 |
| 12 | 20.6 | 185.1 | 13.1 | 8.97 | 0.63 | 0.32 | 0.77 |
| 13 | 89.2 | 4928.8 | 60.3 | 55.28 | 0.68 | 1.97 | 0.82 |
| 14 | 49.0 | 776.3 | 78.7 | 15.83 | 1.61 | 0.56 | 1.96 |
| 15 | 1.0 | 37.4 | 0.7 | 37.73 | 0.71 | 1.35 | 0.86 |
| 16 | 496.8 | 39245.6 | 302.1 | 78.99^{* 1} | 0.61 | — | 0.74 |
| 17 | 4.4 | 50.3 | 5.6 | 11.50 | 1.27 | 0.41 | 1.55 |
| 18 | 13.2 | 356.4 | 11.5 | 27.01 | 0.87 | 0.96 | 1.06 |
| 19 | 0.43 | 10.5 | 0.00 | 24.50 | 0.00 | 0.87 | 0.00 |
| average | — | — | — | 28.02 | 0.82 | 1.00 | 1.00 |
| N(B/S) | | | | | | | |

^{* 1}data was excluded from calculation for average
B/S value after normalization of 532-3p = (B/S) ÷ 28.02
B/S value after normalization of 423-3p = (B/S) ÷ 0.82

The average values of the content ratios N (532-3p) and N (423-3p) for each biomarker were calculated based on data in Table 3. In the calculation of the average for N (532-3p), Sample No. 16 was excluded. The calculation results are as follows.

hsa-miR-532-3p

$$N ( 532 - 3 p ) = \{ sum of [ {B}_{532 - 3 p} / S ] of 18 healthy subjects \} / 18 = 28.02$$

hsa-miR-423-3p

$$N ( 423 - 3 p ) = \{ sum of 19 healthy subjects [ {B}_{423 - 3 p} / S ] / 19 = 0.82$$

For each specimen sample, the value normalized by the average values (called as “normalized B/S”) is also shown in Table 3. The normalization refers to conversion when the average value is 1. The normalized value is obtained for each marker by dividing the respective content ratio of each specimen by the average value. The normalized value for hsa-miR-532-3p is obtained by dividing the content ratio [B₅₃₂₋₃ₚ/S] by the average value 28.02 of N (532-3p), and the normalized value for hsa-miR-423-3p is obtained by dividing the content [B423-3p/S] by the average value 0.82 of N (423-3p).

### (2) Calculation of P (B/S) and Setting of Cut-Off Value

For RNA-containing samples for measurement prepared from specimens of tuberculosis patients (Nos. 21 to 39) by Preparation Method 2, the contents of the standard internal microRNA (hsa-miR-423-5p) and the biomarkers selected from the second group (hsa-miR-532-3p and hsa-miR-423-3p) were calculated from the Ct value obtained using the real-time PCR. Next, the content ratio [B/S] of the content (B) of each biomarker to the content(S) of the hsa-miR-423-5p was calculated. Furthermore, Table 4 shows the calculation results of normalized values (normalized B/S) obtained by dividing the calculated [B/S] of each specimen by the average value of N (B/S) in the healthy subjects.

TABLE 4

| infected | | | | | | | |
|---|---|---|---|---|---|---|---|
| subject | content | | | B/S | | normalized B/S | |
| No. | 423-5p | 532-3p | 423-3p | 532-3p | 423-3p | 532-3p | 423-3p |
| 21 | 29.7 | 110.5 | 21.8 | 3.72 | 0.73 | 0.13 | 0.89 |
| 22 | 4.3 | 0.0 | 1.0 | 0.00 | 0.24 | 0.00 | 0.30 |
| 23 | 12.3 | 406.4 | 5.2 | 32.94^{* 1} | 0.42 | 1.18^{* 1} | 0.51 |
| 24 | 15.9 | 186.1 | 4.6 | 11.71 | 0.29 | 0.42 | 0.35 |
| 25 | 0.9 | 1.0 | 0.0 | 1.09 | 0.00 | 0.04 | 0.00 |
| 26 | 10.6 | 10.1 | 0.6 | 0.95 | 0.06 | 0.03 | 0.07 |
| 27 | 4.3 | 0.0 | 0.1 | 0.00 | 0.03 | 0.00 | 0.04 |
| 28 | 7.8 | 0.0 | 2.3 | 0.00 | 0.29 | 0.00 | 0.36 |
| 29 | 2.1 | 10.0 | 2.4 | 4.79 | 1.17 | 0.17 | 1.42 |
| 30 | 8.6 | 5.6 | 8.8 | 0.65 | 1.02 | 0.02 | 1.24 |
| 31 | 1.9 | 11.2 | 0.3 | 6.02 | 0.17 | 0.21 | 0.20 |
| 32 | 55.6 | 35.9 | 3.3 | 0.65 | 0.06 | 0.02 | 0.07 |
| 33 | 2.1 | 17.2 | 1.2 | 8.07 | 0.59 | 0.29 | 0.71 |
| 34 | 43.0 | 549.2 | 33.3 | 12.77 | 0.78 | 0.46 | 0.94 |
| 35 | 17.1 | 19.2 | 10.2 | 1.12 | 0.60 | 0.04 | 0.73 |
| 36 | 2.3 | 5.5 | 1.1 | 2.32 | 0.47 | 0.08 | 0.57 |
| 37 | 14.3 | 333.8 | 14.7 | 23.41 | 1.03 | 0.84 | 1.25 |
| 38 | 8.5 | 18.6 | 4.0 | 2.18 | 0.46 | 0.08 | 0.56 |
| 39 | 0.5 | 0.0 | 0.0 | 0.00 | 0.00 | 0.00 | 0.00 |
| average | — | — | — | 5.92 | 0.44 | 0.22 | 0.54 |
| P(B/S) | | | | | | | |

^{* 1}data was rejected by the test
B/S value after normalization of 532-3p = (B/S) ÷ 28.02
B/S value after normalization of 423-3p = (B/S) ÷ 0.82

As can be seen from Tables 3 and 4, where the biomarker miR532-3p was used, the normalized value fell within the range from 0.32 to 1.97 in the healthy subjects, whereas within the range from 0 to 0.84 in the tuberculosis infected subjects. No. 23 was not taken into consideration due to rejection by the test. Where the biomarker miR423-3p was used, the value of normalized [B/S] in the healthy subjects fell within the range from 0.00 to 1.96, whereas within the range from 0 to 1.42 in the tuberculosis infected subjects.

The average value of P (B₅₃₂₋₃ₚ/S) is 5.92, and the average value of P (B₄₂₃₋₃ₚ/S) is 0.44, both of which are significantly lower than the average values of N (532-3p) and N (423-3p). Where comparison is made in normalized B/S, the normalized values for respective biomarkers are 0.22 and 0.54, relative to 1.0 for the healthy subjects. These normalized values showed statistically and significantly low comparing with that of healthy subjects (significance levels P<0.0001 and P<0.0031). Therefore, these biomarkers selected from the second group would be effective.

ROC analysis was performed using statistical analysis software EZR (Reference: Bone Marrow Transplantation (2013) 48,452-458) to determine cut-off values. The obtained cut-off values are as follows. In setting of the cut-off values for the biomarker miR532-3p, data of No. 23 was not taken into consideration due to rejection by the test.
Cut-off value for the marker miR532-3p: 0.46
Cut-off value for the marker miR423-3p: 0.73

### (3) Verification of Screening Method

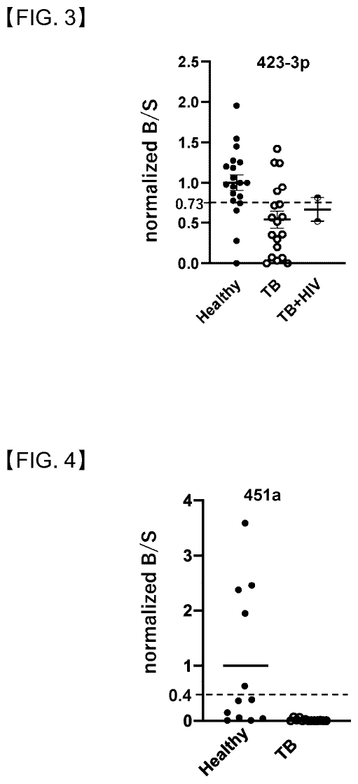

The normalized [B/S] ratios indicated in Tables 3 and 4 are plotted in FIGS. 2 and 3 respectively. In FIGS. 2 and 3, data rejected by the test is not plotted.

In the figures, the vertical axis represents the normalized content ratio [B/S] for each biomarker. Thus, 1.0 on the vertical axis indicates the average value of N (B/S).

As illustrated in FIG. 2 (diagram regarding miR532-3p), where the cut-off value was set to 0.46, about 94% of the specimens were included within the range below the cut-off value based on which specimens were categorized as positive for tuberculosis infection.

Also as illustrated in FIG. 3 (miR423-3p), where the cut-off value was set to 0.73, about 74% of the specimens were included within the range of below the cut-off value based on which specimens were categorized as positive for tuberculosis infection.

From these, it is confirmed that the specimens can be differentiated between tuberculosis infection-positive samples and negative samples based on the cut-off value in the content ratio [B/S] for the biomarker. The [B/S] is a ratio in contents of the biomarker to hsa-miR-423-5p (standard microRNA).

This suggest that, according to the screening method of the present disclosure, highly possibly tuberculosis infection-positive samples can be easily screened by simply detecting and quantifying a specific number of miRNAs.

When using a biomarker chosen from either the first or second group, use of multiple biomarkers from the group can lessen false negatives/false positives.

For example, false positives can be avoided by determining tuberculosis infection-positive only when the results of comparison with the cut-off value match for all the markers.

On the other hand, from the viewpoint of reducing false negatives for the application of medical examination, it may be preferable to determine the likelihood of positive for tuberculosis infection based on the result of comparison with the cut-off value for at least one marker to be used. In an exemplary case of subject No. 29, the sample No. 29 may be determined as tuberculosis infection-negative based on the normalized B/S value for the biomarker hsa-miR423-3P of 1.42, however, this determination is false negative. On the other hand, the sample No. 29 may be categorized into possibly tuberculosis infection-positive based on the comparison result for the marker 523-3p whose normalized B/S value for this marker is 0.17, which is below the cut-off value of 0.46. Thus, the determination can be avoided from the false negative.

### [Verification 2 of Biomarker by Screening Method of the Present Invention]

The RNA-containing samples for measurement were prepared from the healthy subjects (Nos. 1 to 19) and the tuberculosis patients (Nos. 21 to 39) by Preparation method 2 in the same manner as in No. 1. The hsa-miR-451a (ID: 37) as the selected biomarker from the second group was amplified by the real-time PCR and Ct value was obtained, and the contents were calculated based on the Ct value in the same manner as in Preparation method 1. The ratio [B/S] of the content (B) of each biomarker to the content(S) of the standard miRNA (hsa-miR-423-5p) was calculated.

An average value of the B/S in the healthy subjects was calculated, and B/S for each biomarker was divided by the average value for normalization. That is, the average value (N (B/S)) after normalization for the healthy subjects is 1.00. A scatter diagram of the normalized B/S is shown in FIG. 4. The average value N (451a) of the B/S for the healthy subjects was 0.066, and the average value P (451a) of the B/S value for the tuberculosis patients was 0.0000089.

The healthy subjects had a normalized B/S value ranging from 0 to 3.56, whereas the tuberculosis patients had a normalized B/S value for the hsa-miR-451a ranging from 0 to 0.00072, and the average value thereof was 0.00014. As shown in FIG. 4, even when the cut-off value 0.4 is adopted, the normalized B/S values for samples of the tuberculosis patients were below the cut-off value. Thus, the samples can be determined as highly possibly positive for tuberculosis infection.

On the other hand, where hsa-miR451a is used for the biomarker, a sample may be classified into positive based on a small normalized B/S value for hsa-miR451a, however, may be determined as highly possibly false positive unless the normalized B/S values for the other two markers are below the cut-off value. In an exemplary case, a sample of a certain healthy subject having the normalized B451a/S value of 0 may be classified into positive, however, where the same sample has normalized B532-3P/S of 0.46 (cut-off value: 0.46) and normalized B423-3p/S of 2.44 (cut-off value: 0.73), the sample can be determined as highly possibly false positive.

### [Verification 3 of Biomarker by Screening Method of the Present Invention]

RNA-containing samples for measurement were prepared from urine specimens (24 healthy subjects and 39 patients infected with active tuberculosis) by Preparation method 2. The urine specimens were obtained separately from the specimens used in Verification 1 and 2. The real-time PCR was performed on the prepared RNA samples for measurement in accordance with Verification 1 of biomarker to calculate the contents of the standard internal microRNA (hsa-miR-423-5p) and the biomarkers selected from the second group (i.e. hsa-miR-532-3p and hsa-miR-423-3p). Next, the ratio [B/S] of the content (B) of each biomarker to the content(S) of the hsa-miR-423-5p was calculated. Further, for each specimen sample, the value was normalized by use of the average value obtained for the healthy subjects.

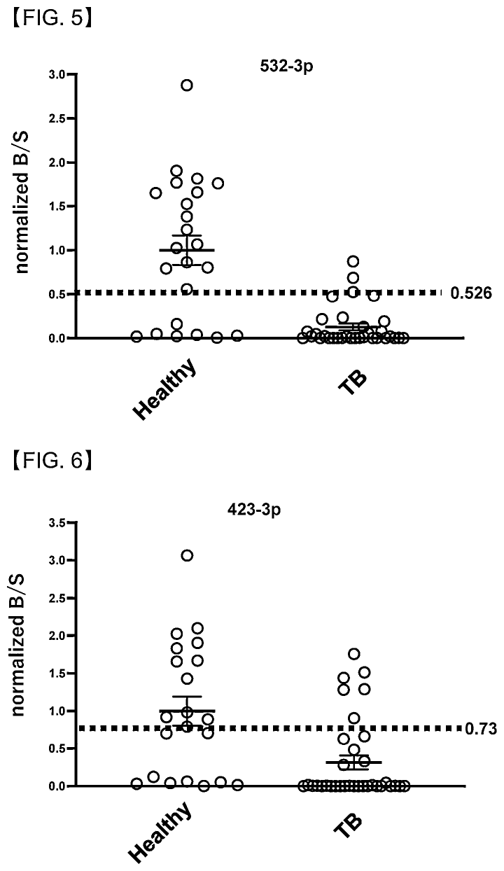

FIG. 5 illustrates a scatter diagram of normalized [B₅₃₂₋₃ₚ/S], and FIG. 6 illustrates a scatter diagram of normalized [B₄₂₃₋₃ₚ/S].

The cut-off value of [B₅₃₂₋₃ₚ/S] was set to 0.526 and the cut-off value of [B₄₂₃₋₃ₚ/S] was set to 0.73. The average value of [B₅₃₂₋₃ₚ/S] for normalization was calculated using data from subject infected with tuberculosis (n=34). In this calculation of the average value, any values significantly deviating from the population or originating from samples with too low expression levels to amplify with a particular number of cycles in real-time PCR were excluded.

The results of classification between positive and negative based on the comparison with the cut-off values were utilized for the calculation of sensitivity and specificity. The following table is referred to the calculation.

TABLE 5

| | subject infected with | subject non-infected with |
|---|---|---|
| | tuberculosis | tuberculosis |
| determination | (a) positive (true) | (b) positive (false) |
| based on | (c) negative (false) | (d) negative (true) |
| comparison with | | |
| the cut-off value | | |

The “sensitivity” is a proportion of determination that a specimen (true positive) infected with tuberculosis is correctly determined as a sample infected with tuberculosis, and is calculated by the following formula.

$$Sensitivity = a / ( a + c )$$

The “specificity” is a proportion of determination that a specimen not suffering from tuberculosis (true negative) is correctly determined as a sample not suffering from tuberculosis, and is calculated by the following formula.

$$Specificity = d / ( b + d )$$

Where miR532-3p was used as the biomarker, the sensitivity was 0.939 and the specificity was 0.696. Where miR423-3p was used as the biomarker, the sensitivity was 0.818 and the specificity was 0.571.

Therefore, where the determination as positive is made based on the comparison results of both [B₅₃₂₋₃ₚ/S] and [B₄₂₃₋₃ₚ/S] with the respective cut-off value, the sample can be determined as positive for tuberculosis infection at highly possibility. Thus, the number of false positive samples can be reduced.

From the above, it could be confirmed that the differentiation between the presence or absence of the tuberculosis infection is reproducible where the above-mentioned biomarkers and the standard miRNA are used; that categorizing based on normalized value with the standard miRNA hsa-miR-423-5p exhibits high sensitivity and specificity; and that combination of determination results of multiple biomarkers can ensure an accurate differentiation of samples between positive and negative for tuberculosis infection.

### INDUSTRIAL APPLICABILITY

In the screening method for tuberculosis infection of the present invention, urine specimens are used to classify the presence or absence of active tuberculosis infection. Urine is a less invasive specimen for a subject and can be easily collected from the subject. Moreover, it does not require culturing or similar process prior to determining tuberculosis infection. Therefore, this screening method of the invention is useful in areas such as developing countries with limited equipment, as it can rapidly provide determination result.

1-8. (canceled)

9. A method for screening a tuberculosis-infected sample by detecting one biomarker miRNAs selected from miRNAs listed in Table 1 (sequence ID: 2 to ID: 33), and hsa-miR-532-3p (sequence ID: 49) and hsa-miR-423-3p (sequence ID: 56), contained in a urine-derived sample from a subject, the method comprising:
obtaining a ratio [B/S] of content B of the biomarker miRNA to content S of hsa-miR-423-5p in a given urine-derived sample;
comparing the obtained ratio [B/S] of the urine-derived sample with a cut-off value, wherein the cut-off value is a value based on N (B/S) and P [B/S], wherein the N (B/S) represents a ratio of content B of the biomarker miRNA to content S of hsa-miR-423-5p in a urine-derived sample from a healthy/normal subject while P [B/S] represents a corresponding ratio in a urine-derived sample from a tuberculosis infected subject; and
classifying the given urine-derived sample from the subject into a tuberculosis infection-positive or a tuberculosis-negative based on the comparison result:
TABLE 1

| sequence | | |
|---|---|---|
| No. | name | base sequence |
| 2 | hsa-miR-196a-5p | uagguaguuucauguuguuggg |
| 3 | hsa-miR-500a-3p | augcaccugggcaaggauucug |
| 4 | hsa-miR-424-5p | cagcagcaauucauguuuugaa |
| 5 | hsa-miR-4508 | gcggggcugggcgcgcg |
| 6 | hsa-miR-1180-3p | uuuccggcucgcgugggugugu |
| 7 | hsa-miR-92b-3p | uauugcacucgucccggccucc |
| 8 | hsa-miR-181a-5p | aacauucaacgcugucggugagu |
| 9 | hsa-miR-4459 | ccaggaggcggaggagguggag |
| 10 | hsa-miR-199a-5p | cccaguguucagacuaccuguuc |
| 11 | hsa-miR-1972 | ucaggccaggcacaguggcuca |
| 12 | hsa-miR-7641 | uugaucucggaagcuaagc |
| 13 | hsa-miR-4787-5p | gcggggguggcggcggcauccc |
| 14 | hsa-miR-373-3p | gaagugcuucgauuuuggggugu |
| 15 | hsa-miR-1268a | cgggcguggugguggggg |
| 16 | hsa-miR-6858-5p | gugaggaggggcuggcagggac |
| 17 | hsa-miR-1273d | gaacccaugagguugaggcugc |
| | | agu |
| 18 | hsa-miR-1285-3p | ucugggcaacaaagugagaccu |
| 19 | hsa-miR-4497 | cuccgggacggcugggc |
| 20 | hsa-miR-6126 | gugaaggcccggcggaga |
| 21 | hsa-miR-335-5p | ucaagagcaauaacgaaaaaugu |
| 22 | hsa-miR-663a | aggcggggcgccgcgggaccgc |
| 23 | hsa-miR-4466 | gggugcgggccggcgggg |
| 24 | hsa-miR-30c-2- | cugggagaaggcuguuuacucu |
| | 3p | |
| 25 | hsa-miR-502-3p | aaugcaccugggcaaggauuca |
| 26 | hsa-miR-500a-5p | uaauccuugcuaccugggugaga |
| 27 | hsa-miR-6723-5p | auaguccgaguaacgucggggc |
| 28 | hsa-miR-150-3p | ccgguacaggccugggggacag |
| 29 | hsa-miR-1273g- | accacugcacuccagccugag |
| | 3p | |
| 30 | hsa-miR-514b-5p | uucucaagagggaggcaaucau |
| 31 | hsa-miR-1299 | uucuggaauucugugugaggga |
| 32 | hsa-miR-362-3p | aacacaccuauucaaggauuca |
| 33 | hsa-miR-1285-5p | gaucucacuuuguugcccagg |

10. The method for screening according to claim 9, wherein the cut-off value is a value capable of statistically differentiating between a tuberculosis-infected population and a healthy/normal population, based on a ratio B/S (P (B/S)) of the tuberculosis-infected population and an average of ratio B/S (N (B/S)) of the healthy/normal population.

11. The method for screening according to claim 9, wherein the classifying step is performed by a first sorting using a miRNA listed in Table 1 as a first biomarker and a second sorting using hsa-miR-532-3p (sequence ID: 49) and hsa-miR-423-3p (sequence ID: 56) as second biomarkers,
wherein the second sorting step is a step that the given urine-derived sample with a [B/S] ratio below the cut-off value is sorted as tuberculosis-infected sample, and
wherein the given urine-derived sample is determined as a tuberculosis-positive if the given urine-derived sample is sorted as a tuberculosis-infected sample in both of the first and the second sorting.

12. A method for screening a tuberculosis-infected sample comprising:
measuring respective contents of hsa-miR-423-5p, and biomarker miRNAs of hsa-miR-532-3p (sequence ID: 49) and hsa-miR-423-3p (sequence ID: 56), contained in a given urine-derived sample from a subject;
obtaining a ratio [B/S] of content B of each of the biomarker miRNAs to content S of hsa-miR-423-5p; and
comparing the obtained ratio [B/S] of the given urine-derived sample, with a cut-off value wherein the cut-off value is a value set based on N (B/S) and P [B/S], wherein the N (B/S) represents a ratio of content B of the biomarker miRNA to content S of hsa-miR-423-5p in a urine-derived sample from a healthy/normal subject while P [B/S] represents a corresponding ratio in a urine-derived sample from a tuberculosis infected subject; and
classifying the given urine-derived sample as a tuberculosis infection-positive if a comparison result is consistent across that at least one or all of the biomarkers are below the respective cut-off value.

13. A probe set used for screening a positive active tuberculosis sample among samples each derived from urine of a given subject, the probe set comprising:
a standard nucleotide probe capable of hybridizing to hsa-miR-423-5p under stringent conditions and labeled for detection; and
at least one probe selected from the group consisting of nucleotide probe 1 and nucleotide probe 2 as indicated below:
Probe 1: a nucleotide probe capable of hybridizing to one or more nucleotides selected from sequences of ID2 to ID33 under stringent conditions and each labeled for detection;
Probe 2: a nucleotide probe capable of hybridizing to at least two nucleotides selected from the group consisting of sequences ID37, ID49, and ID56 under stringent conditions and each labeled for detection.

14. A probe set used for screening a positive sample of active tuberculosis among samples each derived from urine of a given subject, the probe set comprising:
a standard nucleotide probe capable of hybridizing to hsa-miR-423-5p under stringent conditions and labeled for detection; and
Probe 3: a combination of a first and second nucleotide probes for biomarkers, the first probe being capable of hybridizing to a nucleotide of ID49 and the second probe being capable of hybridizing to a nucleotide ID56, under stringent conditions and each probe being labeled for detection.
